(19) United States
(12) Patent Application Publication
(10) Pub. No.: US 2023/0274186 A1
(43) Pub. Date: Aug. 31, 2023

(54) Determinations of Characteristics from Biometric Signals

(71) Applicant: Hewlett-Packard Development Company, L.P., Spring, TX (US)

(72) Inventors: Jishang Wei, Guilford, CT (US); Rafael Ballagas, Palo Alto, CA (US)

(73) Assignee: Hewlett-Packard Development Company, L.P., Spring, TX (US)

(21) Appl. No.: 18/043,321

(22) PCT Filed: Sep. 8, 2020
(86) PCT No.: PCT/US2020/049694

§ 371 Date: Feb. 27, 2023

(51) Int. Cl. G06N 20/00; G06F 3/01

(52) U.S. Cl. CPC G06N 20/00; G06F 3/015

(57) ABSTRACT

An example system includes a plurality of biometric sensors. The system also includes a first classifier engine to produce a first latent space representation of a first signal from a first biometric sensor of the plurality of biometric sensors. The system includes a second classifier engine to produce a second latent space representation of a second signal from a second biometric sensor of the plurality of biometric sensors. The system includes an attention engine to weight the first latent space representation and the second latent space representation based on correlation among latent space representations. The system includes a final classifier engine to determine a characteristic of a user based on the weighted first and second latent space representations.

### BACKGROUND

A system may measure biometric signals of a user. For example, the system may include a head mounted display able to produce a virtual reality (VR) experience, an augmented reality (AR) experience, a mixed reality (MR) experience, or the like. VR, AR, and MR may be collectively referred to as extended reality (XR). The system may also include controllers, haptic feedback devices, or the like. The system may measure biometric signals from the user. For example, the head mounted display, the controller, or the haptic feedback devices may measure the biometric signals.

### BRIEF DESCRIPTION OF THE DRAWINGS

FIG. 1 is a block diagram of an example system to determine characteristics from biometric signals.

FIG. 2 is a block diagram of another example system to determine characteristics from biometric signals.

FIG. 3 is a flow diagram of an example method to determine characteristics from biometric signals.

FIG. 4 is a flow diagram of another example method to determine characteristics from biometric signals.

FIG. 5 is a block diagram of an example computer-readable medium including instructions that cause a processor to determine characteristics from biometric signals.

FIG. 6 is a block diagram of another example computer-readable medium including instructions that cause a processor to determine characteristics from biometric signals.

### DETAILED DESCRIPTION

A system, such as an XR system, may measure various biometric signals. For example, the biometric signals may be a heart rate signal (e.g., a photoplethysmography (PPG) signal, an electrocardiogram (ECG) signal, etc.), a galvanic skin response signal, a pupillometry signal, an eye tracking signal, an electromyography (EMG) signal, a respiration rate signal, a blood pressure signal, or the like. The various signals may be indicative of a state of a user. The system may adjust a user experience based on the state of the user. For example, the system may detect a cognitive load (e.g., is the user bored, overwhelmed, etc.) of a user and adjust the experience to produce an ideal cognitive load. The system may detect the user's level of change blindness and modify a scene based on the change blindness exceeding a threshold.

A system that receives multiple biometric signals may make a decision about the state of the user based on the multiple signals. For example, the system may fuse the signals to make the decision about the state of the user. There are various ways for the system to fuse the signals. In an example, the system may perform decision level fusion. To perform decision level fusion, the system may combine multiple decisions about the state of the user each made from an individual signal. For example, each signal may be analyzed by a corresponding neural network to make a decision about the state of a user. The decisions may be combined to reach a final decision about the state of the user, for example, by averaging the decisions, selecting a median decision, consulting a lookup table, or the like.

In an example, the system may perform feature level fusion. To perform feature level fusion, the system may convert each signal into a feature vector and combine the feature vectors. The system may make a decision based on the combined feature vectors. For example, a single neural network may make a decision about the state of the user based on the combined feature vectors. The feature vectors for the various signals may be concatenated, and the concatenated vector may be used as an input to the neural network.

Using decision level fusion and feature level fusion may each have disadvantages. When performing decision level fusion, information about correlations among the signals may be lost because the final decisions may be fused without any additional information about the signals and with the signals otherwise processed separately. Accordingly, the final decision may not be as accurate as it could be if the correlations and relationships among the signals were considered. When performing feature level fusion, the decisions may not be robust against signal loss. If a signal goes offline or experiences a temporary disruption, the neural network may be unable to make an accurate decision regarding the state of the user. For example, user movement, blinking, etc. may disrupt measurements by biometric sensors. The biometric sensors may experience with enough frequency that the decision errors may affect the user experience. Accordingly, decisions about the user state could be improved by providing for decisions that leverage the correlations and relationships among multiple signals while being robust to the loss of individual signals.

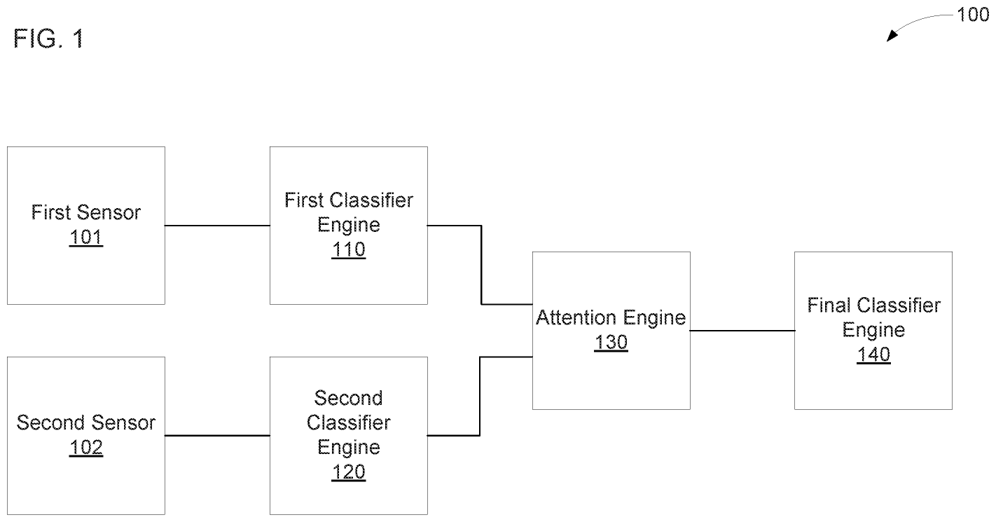

FIG. 1 is a block diagram of an example system 100 to determine characteristics from biometric signals. The system 100 may include a plurality of biometric sensors, including a first biometric sensor 101 and a second biometric sensor 102. As used herein, the “biometric sensor” refers to a sensor that measures a characteristic of a biological entity, such as a human, that changes based on voluntary or involuntary biological functions of the biological entity. In some examples, the first biometric sensor 101 may measure a first characteristic of a user of the system 100, and the second biometric sensor 102 may measure a second characteristic of the user different from the first.

The system 100 may include a first classifier engine 110 and a second classifier engine 120. As used herein, the term “engine” refers to hardware (e.g., analog or digital circuitry, a processor, such as an integrated circuit, or other circuitry) or a combination of software (e.g., programming such as machine- or processor-executable instructions, commands, or code such as firmware, a device driver, programming, object code, etc.) and hardware. Hardware includes a hardware element with no software elements such as an application specific integrated circuit (ASIC), a Field Programmable Gate Array (FPGA), etc. A combination of hardware and software includes software hosted at hardware (e.g., a software module that is stored at a processor-readable memory such as random-access memory (RAM), a hard-disk or solid-state drive, resistive memory, or optical media such as a digital versatile disc (DVD), and/or executed or interpreted by a processor), or hardware and software hosted at hardware. The first classifier engine 110 may produce a first latent space representation of a first signal from the first biometric sensor. The second classifier engine may produce a second latent space representation of a second signal from the second biometric sensor. As used herein, the term “latent space representation” refers to a multidimensional representation of a signal in a multidimensional space. The latent space representation may include a feature vector indicative of features in the signal and may be a compressed representation of the signal. The first or second classifier engine may each include machine learning model, such as a neural network, to generate the first or second latent space representation.

The system 100 may also include an attention engine 130 to weight the first latent space representation and the second latent space representation based on correlation among latent space representations. For example, the attention engine 130 may calculate a weight for each latent space representation corresponding to how well correlated that latent space representation is with other latent space representations. The attention engine 130 may apply the calculated weight for a particular latent space representation to that latent space representation.

The system may include a final classifier engine 140. The final classifier engine 140 may determine a characteristic of a user based on the weighted first and second latent space representations. For example, the final classifier engine 140 may include a machine learning model, such as a neural network, to determine the characteristic. The machine learning model may have been trained to determine the characteristic based on the weighted latent space representations.

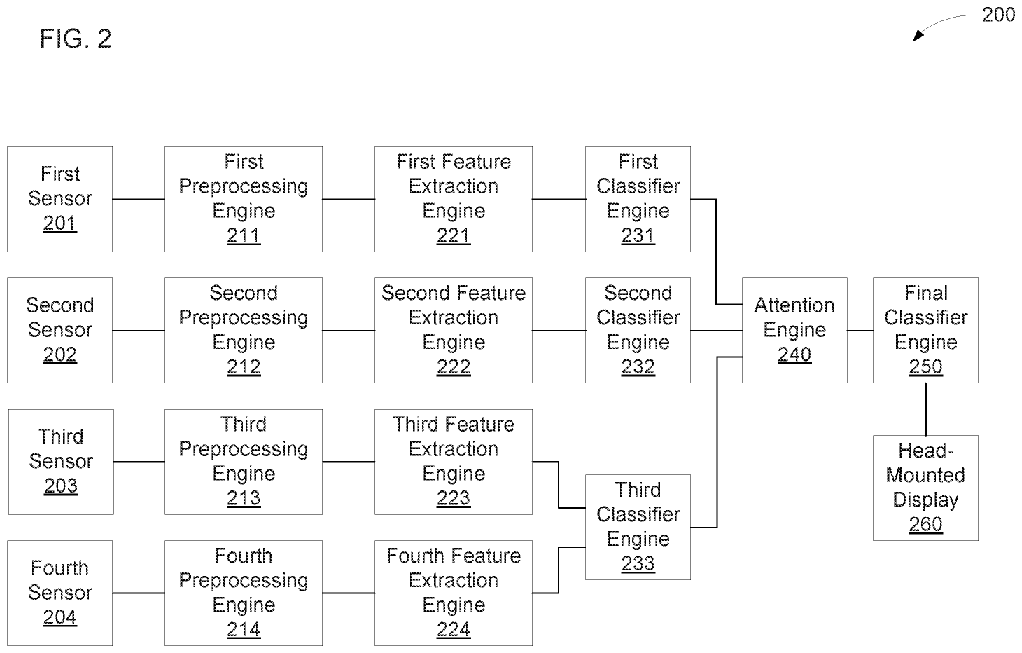

FIG. 2 is a block diagram of another example system 200 to determine characteristics from biometric signals. The system 200 may include a plurality of biometric sensors, including a first biometric sensor 201, a second biometric sensor 202, a third biometric sensor 203, and a fourth biometric sensor 204. The sensors 201-204 may be sensors in a headset, controllers, clothing, a backpack, or the like worn or held by a user of the system. The sensors 201-204 may measure various characteristics of the user. Each sensor 201-204 may generate a signal, such as a heart rate signal (e.g., a photoplethysmography (PPG) signal, an electrocardiogram (ECG) signal, etc.), a galvanic skin response signal, a pupillometry signal, an eye tracking signal, an electromyography (EMG) signal, a respiration rate signal, a blood pressure signal, or the like.

The system 200 may also include a plurality of preprocessing engines, including a first preprocessing engine 211, a second preprocessing engine 212, a third preprocessing engine 213, and a fourth preprocessing engine 214. The preprocessing engines 211-214 may convert each signal to a time series. The preprocessing engines 211-214 may prepare the signals so that features can be extracted from them. For example, the preprocessing engines 211-214 may remove noise from the signal, detrend the signal, or the like. The preprocessing engines 211-214 may process the non-stationary signals to cause them to more closely resemble stationary signals. The type of preprocessing may depend on the particular signal.

The system 200 may include a plurality of feature extraction engines 221-224, including a first feature extraction engine 221, a second feature extraction engine 222, a third feature extraction engine 223, and a fourth feature extraction engine 224. Each feature extraction engine 221-224 may generate a feature vector based on the preprocessed signal from the corresponding preprocessing engine 211-214. For example, the feature extraction engine 221-224 may determine a feature vector based on a time series generated by the preprocessing engine 211-214. Various aspects of the preprocessed signal may be used as features depending on the particular signal. For example, the features may include mean, variation, or the like. The feature extraction engine 221-224 may convert the signal to the frequency domain and include frequency domain information. For a domain specific signal, the feature extraction engine 221-224 may calculate a meaningful value for the application. For example, the feature extraction engine 221-224 may calculate a blinking rate based on an eye tracking or pupillometry signal.

The system 200 may include a plurality of classifier engines 231-233 to receive the feature vectors from the feature extraction engines 221-224 and generate latent space representations based on the feature vectors. In some examples, the classifier engines 231-233 may have been trained to identify a characteristic of the user independent of the other classifier engines 231-233 and independent of the attention engine 240 and the final classifier engine 250. The classifier engines 231-233 may include machine learning models, such as neural networks, that are trained to identify the characteristics of the user based on examples of various signals/feature vectors corresponding each characteristic. The output of each classifier engine 231-233 may be a vector of soft determinations. As used herein, the term “soft determinations” refers to values indicative of how likely each determination is true. For example, the vector may be a softmax vector with each value in the vector indicative of the probability of the user having the characteristic corresponding to the value. That is, the latent space representation may be a vector of softmax values.

As can be seen in the example of FIG. 2, the classifier engines may generate the latent space representation based on a feature vector associated with a single sensor (e.g., the first and second classifier engines 231-232) or based on feature vector associated with multiple sensors (e.g., the third classifier engine 233). The third and fourth feature extraction engines 223,224 may generate third and fourth feature vectors respectively. The third and fourth feature vectors may be concatenated, and the third classifier engine 233 may generate a third latent space representation based on the concatenation of the third and fourth feature vectors. Other variations are contemplated, such as, a feature vector being provided to multiple classifier engines (with or without concatenation to other feature vectors) or generation of multiple different feature vectors from a single sensor, which may be provided to a single classifier or multiple classifiers.

The system 200 may include an attention engine 240. The attention engine 240 may receive the first, second, and third latent space representations from the classifier engines 231-233. The attention engine 240 may weight the first, second, and third latent space representations based on correlations among the latent space representations. For example, a first latent space representation that is more highly correlated with other latent space representations may be more highly weighted than a second latent space vector that is not as highly correlated with other latent space vectors.

In some examples, the attention engine 240 may compute the correlation by computing:

$$S = softmax ( \frac{Q {K}^{T}}{\sqrt{{d}_{k}}} )$$

Where Q and K are matrices formed from stacking the latent space representations output from the classifier engines 231-233, dₖ is the length of the latent space representations, and softmax is the softmax function that normalizes the distribution of the input. The attention engine 240 may weight the latent space representations by computing:

Weighted Latent Space Representations=S*V

Where V is a matrix formed by stacking the latent space representations output from the classifier engines 231-233. V may equal K, which may equal Q.

The attention engine 240 may further weight and combine the weighted latent space representations based on an individual attention model. The attention engine 240 may compute the weights by first computing:

hₙ=Wc′ₙ+b

Where c′n is the nth weighted latent space representation, W and b are parameters generated from a training process, and hn is a scalar. The attention engine 240 may compute the weights by then computing:

a₁, a₂, . . . , aₙ=softmax(h₁, h₂, . . . , hₙ)

Where α₁, α₂, . . . , αₙ an are the weights to be applied to the weighted latent space representations. The attention engine 240 may weight and combine the weighted latent space representations by computing:

$$c = {\sum }_{i = 1}^{n} {α}_{i} {c}_{i}^{′}$$

Where c is the latent space representation resulting from the weighting and combining.

The system 200 may include a final classifier engine 250. The final classifier engine 250 may determine a characteristic of a user based on the weighted latent space representations. The final classifier engine 250 may receive the latent space representation output from the attention engine (e.g., the latent space representation c computed above) and compute a final classification from the output. For example, the final classifier engine 250 may be a neural network that includes a single, linear combination layer and produces a softmax output. The output from the final classifier 250 may be a vector with each value in the vector corresponding to a possible state of the characteristic of the user. Each value may indicate the probability of the characteristic of the user being in that state. The final classifier engine 250 may determine the characteristic of the user by determining which vector value is largest and selecting the state corresponding to that vector value.

In examples, the characteristic of the user may be a mental state of the user (e.g., a cognitive load, a perceptual load, etc.), an emotional state of the user, a physical state of the user, or the like. For a system to determine a cognitive load or a perceptual load, the possible states of the characteristic may be a high load, a medium, and a low load or may include more or fewer possible states. For an emotional state of the user, the possible states may include a selected set of emotions (e.g., happy, sad, angry, afraid, bored, etc.). The physical states may include health conditions, physical effort, physical exhaustion, tiredness, stress, etc. For example, the system may be trained to detect particular adverse health conditions. Physical effort, physical exhaustion, tiredness, stress, etc. may be grouped in a predetermined number of buckets, such as high, medium, and low or the like.

The system 200 may include a head-mounted display 260. The system 200 may alter an audio or video output by the head-mounted display 260 based on the determined characteristic of the user. For example, if a cognitive load is too high, the system 200 may alter the audio or video to reduce cognitive load, e.g., by reducing the number or intensity of stimuli in the audio or video. Conversely, if the cognitive load is too low, the system 200 may alter the audio or video to increase the cognitive load. Similarly, if the user is in an undesired emotional or physical state, the system 200 may alter the audio or video in a way predicted to cause the user to reach the desired emotional or physical state. The audio or video may be altered by the head-mounted display 260 or by a separate engine generating the audio or video for rendering by the head-mounted display. The head-mounted display 260 may include display elements to deliver modified video to the user or headphones or speakers to deliver modified audio to the user.

The system 200 performs decision level fusion, but the system 200 also considers the correlations among the signals using the attention engine 240. Accordingly, the system 200 is able to leverage the benefits of decision level fusion while mitigating the disadvantages. The system 200 is robust to signal loss while also being able to consider the correlations and relationships among the signals to produce a more accurate result.

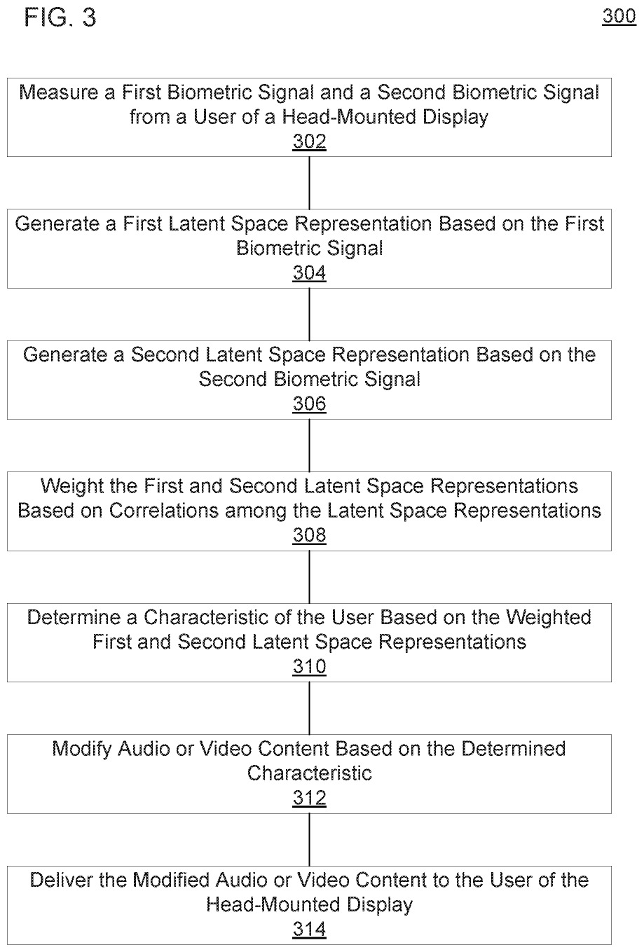

FIG. 3 is a flow diagram of an example method 300 to determine characteristics from biometric signals. A processor may perform elements of the method 300. For illustrative purposes, the method 300 is described in connection with the device of FIG. 2. However, other devices may perform the method in other examples. At block 302, the method 300 may include measuring a first biometric signal and a second biometric signal from a user of a head-mounted display. For example, the first and second sensors 201, 202 may measure the first and second biometric signals in any of the manners previously discussed.

Block 304 may include generating a first latent space representation based on the first biometric signal, and block 306 may include generating a second latent space representation based on the second biometric signal. For example, the first classifier engine 231 may generate the first latent space representation based on the first biometric signal from the first sensor 201 in any of the manners previously discussed. The second classifier engine 232 may generate the second latent space representation based on the second biometric signal from the second sensor 202 in any of the manners previously discussed.

At block 308, the method 300 may include weighting the first latent space representation and the second latent space representation based on correlations among latent space representations. For example, the attention engine 240 may compute correlations among the latent space representations in any of the manner previously discussed. The attention engine 240 may also apply the weights to the first and second latent space representations in any of the manners previously discussed.

Block 310 may include determining a characteristic of the user based on the weighted first and second latent space representations. For example, the final classifier engine 250 may determine the characteristic of the user based on the weighted first and second latent space representations in any of the manners previously discussed. The characteristic may be any of the characteristics previously discussed.

At block 312, the method 300 may include modifying audio or video content based on the determined characteristic. For example, the head-mounted display 260 or a separate rendering engine may modify the audio or video content based on the determined characteristic in any of the manners previously discussed.

Block 314 includes delivering the modified audio or video content to the user of the head-mounted display. For example, the head-mounted display 260 may deliver the modified audio or video content to the user in any of the manners previously discussed.

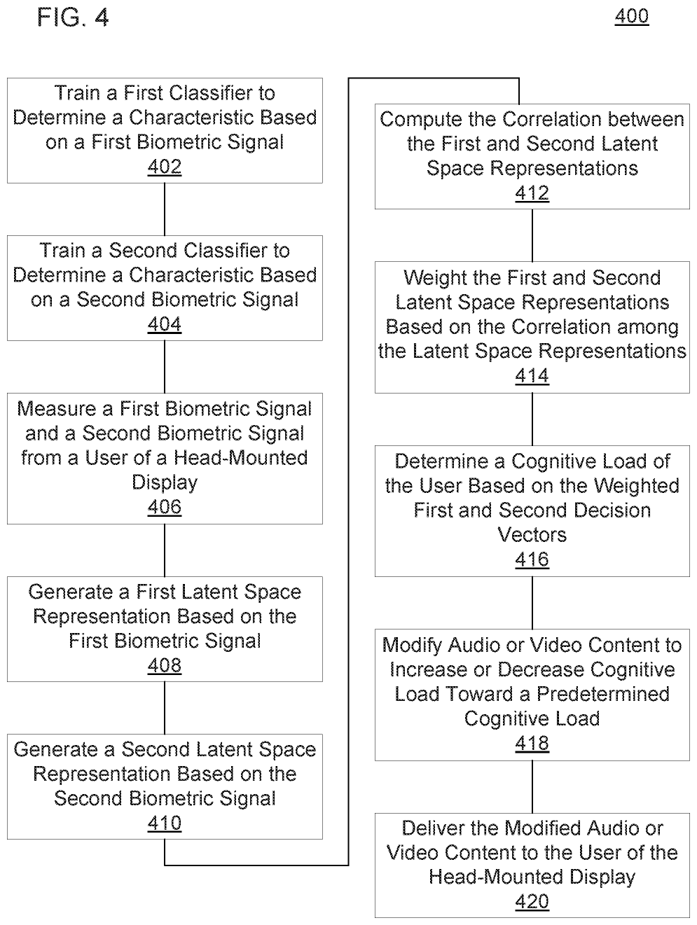

FIG. 4 is a flow diagram of another example method 400 to determine characteristics from biometric signals. A processor may perform elements of the method 400. For illustrative purposes, the method 400 is described in connection with the device of FIG. 2. However, other devices may perform the method in other examples. Block 402 may include training a first classifier to determine the characteristic based on the first biometric signal, and block 404 may include training a second classifier to determine the characteristic based on the second biometric signal. For example, the first and second classifiers may be trained with a labeled training set that includes a plurality of example signals and the state of the characteristic associated with each example signal. In some examples, the first and second classifiers may be neural networks, and classification errors may be backpropagated through the neural networks to adjust the weights of the neural networks. In some examples, a first training engine and a second training engine may train the first and second classifiers respectively.

At block 406, the method 400 may include measuring a first biometric signal and a second biometric signal from a user of a head-mounted display. For example, the first and second sensors 201, 202 may measure the first and second biometric signals in any of the manners previously discussed. Preprocessing and feature extraction may be performed on the first and second biometric signals in any of the manners previously discussed.

Block 408 may include generating a first latent space representation based on the first biometric signal, and block 410 may include generating a second latent space representation based on the second biometric signal. For example, the first classifier engine 231 may generate the first latent space representation based on the first biometric signal using the first classifier in any of the manners previously discussed, and the second classifier engine 232 may generate the second latent space representation based on the second biometric signal using the second classifier in any of the manners previously discussed. The first and second classifiers may include softmax functions to produce the first and second latent space representations.

At block 412, the method 400 may include computing the correlation between the first latent space representation and the second latent space representation. For example, the attention engine 240 may compute the correlation between the first latent space representation and the second latent space representation and any additional latent space representations in any of the manners previously discussed. Block 414 may include weighting the first latent space representation and the second latent space representation based on the correlation among latent space representations. For example, the attention engine 240 may weight the first latent space representation and the second latent space representation based on the correlation among latent space representations in any of the manners previously discussed.

At block 416, the method 400 may include determining a cognitive load of the user based on the weighted first and second latent space representations. For example, the cognitive load may be the characteristic of the user being determined, and the final classifier engine 250 may determine the characteristic in any of the manners previously discussed.

Block 418 may include modifying audio or video content to cause an increase or decrease in the cognitive load of the user toward a predetermined cognitive load. The head-mounted display 260 or a separate rendering engine may modify the audio or video content to cause the increase or decrease in the cognitive load of the user toward the predetermined cognitive load in any of the manners previously discussed. At block 420, the method 400 may include delivering the modified audio or video content to the user of the head-mounted display 260. For example, the head-mounted display 260 may deliver the modified audio or video content to the user in any of the manners previously discussed.

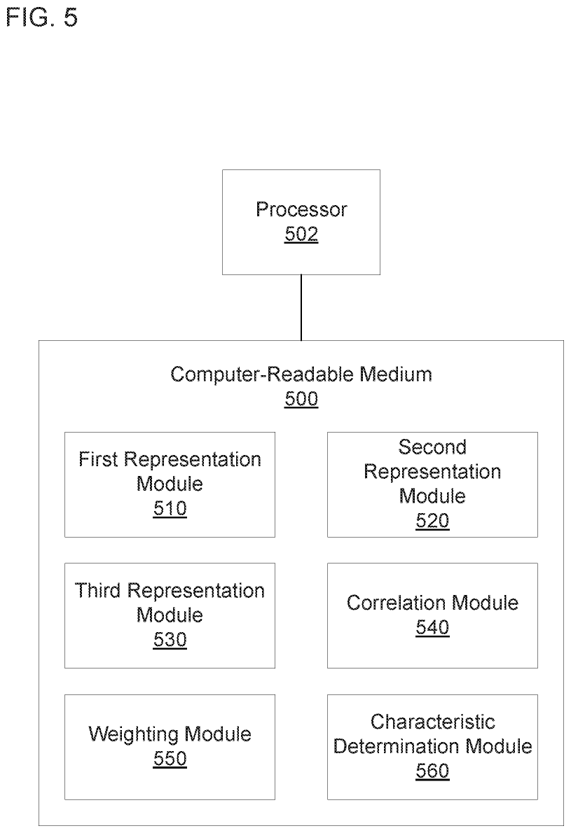

FIG. 5 is a block diagram of an example computer-readable medium 500 including instructions that, when executed by a processor 502, cause the processor 502 to determine characteristics from biometric signals. The computer-readable medium 500 may be a non-transitory computer-readable medium, such as a volatile computer-readable medium (e.g., volatile RAM, a processor cache, a processor register, etc.), a non-volatile computer-readable medium (e.g., a magnetic storage device, an optical storage device, a paper storage device, flash memory, read-only memory, non-volatile RAM, etc.), and/or the like. The processor 502 may be a general-purpose processor or special purpose logic, such as a microprocessor (e.g., a central processing unit, a graphics processing unit, etc.), a digital signal processor, a microcontroller, an ASIC, an FPGA, a programmable array logic (PAL), a programmable logic array (PLA), a programmable logic device (PLD), etc.

The computer-readable medium 500 may include a first representation module 510, a second representation module 520, a third representation module 530, a correlation module 540, a weighting module 550, and a characteristic determination module 560. As used herein, a “module” (in some examples referred to as a “software module”) is a set of instructions that when executed or interpreted by a processor or stored at a processor-readable medium realizes a component or performs a method. The first representation module 510 may include instructions that, when executed, cause the processor 502 to generate a first latent space representation indicative of a characteristic of a user based on a first signal from a first biometric sensor. The second representation module 520 may cause the processor 502 to generate a second latent space representation indicative of the characteristic of a user based on a second signal from a second biometric sensor. The third representation module 530 may cause the processor 502 to generate a third latent space representation indicative of the characteristic of a user based on a third signal from a third biometric sensor. In some examples, the first, second, and third representation modules 510, 520, 530 may implement the first, second, and third classifier engines 231, 232, 233 when executed and may generate the latent space representations indicative of the characteristic of the user based on the signals from the biometric sensors in any of the manners previously discussed.

The correlation module 540 may cause the processor 502 to calculate correlations between the first, second, and third soft latent space representations. The weighting module 550 may cause the processor 502 to weight each of the first, second, and third latent space representations based on the correlations of that latent space representation with the other latent space representations. For example, the correlation module 540 and the weighting module 550 may implement the attention engine 240 when executed. The correlation module 540 may cause the processor 502 to calculate the correlations between the first, second, and third soft latent space representations in any of the manners previously discussed, and the weighting module 550 may cause the processor 502 to weight each of the first, second, and third latent space representations based on the correlations of that latent space representation with the other latent space representations in any of the manners previously discussed.

The characteristic determination module 560 may cause the processor 502 to determine the characteristic of a user based on the weighted first, second, and third latent space representations. In some examples, the characteristic determination module 560 may implement the final classifier engine 250 when executed and may determine the characteristic of the user based on the weighted first, second, and third latent space representations in any of the manners previously discussed.

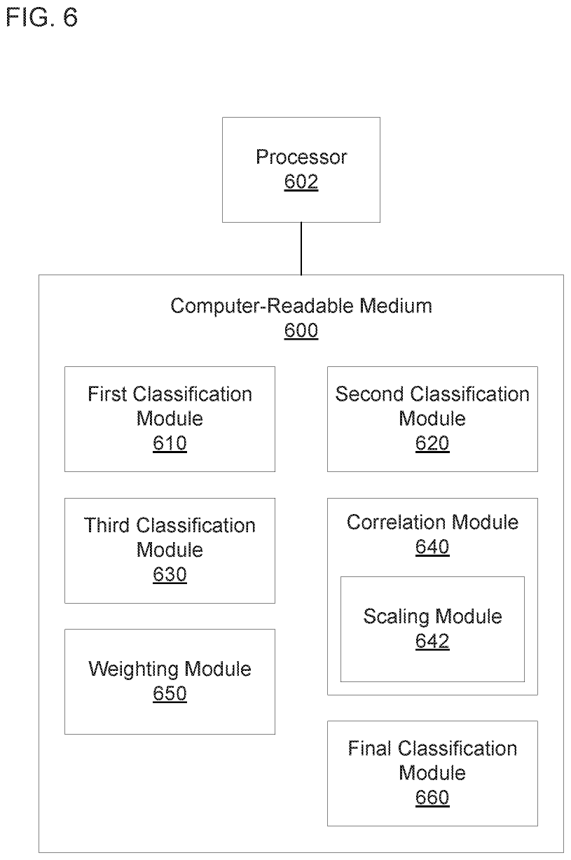

FIG. 6 is a block diagram of another example computer-readable medium 600 including instructions that, when executed by a processor 602, cause the processor 602 to determine characteristics from biometric signals. The computer-readable medium 600 may include a first classification module 610, a second classification module 620, a third classification module 630, a correlation module 640, a scaling module 642, a weighting module 650, and a final classification module 660. The first representation module 610 may include instructions that, when executed, cause the processor 602 to generate a first latent space representation indicative of a characteristic of a user based on a first signal from a first biometric sensor. The second representation module 620 may cause the processor 602 to generate a second latent space representation indicative of the characteristic of a user based on a second signal from a second biometric sensor. The third representation module 630 may cause the processor 602 to generate a third latent space representation indicative of the characteristic of a user based on a third signal from a third biometric sensor. In some examples, the first, second, and third representation modules 610, 620, 630 may implement the first, second, and third classifier engines 231, 232, 233 when executed and may generate the latent space representations indicative of the characteristic of the user based on the signals from the biometric sensors in any of the manners previously discussed. The first, second, or third latent space representations may be soft determinations calculated by first, second, or third classifiers respectively based on the first, second, and third signals.

The correlation module 640 may cause the processor 602 to calculate correlations between the first, second, and third soft latent space representations. For example, the correlation module 640 may cause the processor 602 to stack the first, second, and third latent space representations to form a matrix and to multiply the matrix by its transpose to produce a correlation matrix. The correlation module 640 may include a scaling module 642. The scaling module 642 may cause the processor 602 to scale the correlation matrix to produce a scaled correlation matrix. The correlation module 640 and the scaling module 642 may implement the attention engine 240 when executed and may stack the first, second, and third latent space representations to form the matrix, multiply the matrix by its transpose to produce the correlation matrix, and scale the correlation matrix to produce the scaled correlation matrix in any of the manners previously discussed.

The weighting module 650 may cause the processor 602 to weight each of the first, second, and third latent space representations based on the correlations of that latent space representation with the other latent space representations. For example, the weighting module 650 may cause the processor 602 to multiply the scaled correlation matrix with the matrix formed by stacking the latent space representations. The weighting module 650 may also cause the processor to further weight each of the weighted first, second, and third latent space representations based on values of that representation. In some examples, the weighting module 650 may implement the attention engine 240 when executed and may multiply the scaled correlation matrix with the matrix formed by stacking the latent space representations and further weight each of the weighted first, second, and third latent space representations based on values of that representation in any of the manners previously discussed.

The final classification module 660 may cause the processor 602 to determine the characteristic of a user based on the weighted first, second, and third latent space representations. For example, the final classification module 660 may cause the processor 602 to determine the characteristic based on the further weighted first, second, and third latent space representations. In some examples, the final classification module 660 may implement the final classifier engine 250 when executed and may determine the characteristic based on the further weighted first, second, and third latent space representations in any of the manners previously discussed.

The above description is illustrative of various principles and implementations of the present disclosure. Numerous variations and modifications to the examples described herein are envisioned. Accordingly, the scope of the present application should be determined only by the following claims.

What is claimed is:

1. A system comprising:
a plurality of biometric sensors;
a first classifier engine to produce a first latent space representation of a first signal from a first biometric sensor of the plurality of biometric sensors;
a second classifier engine to produce a second latent space representation of a second signal from a second biometric sensor of the plurality of biometric sensors;
an attention engine to weight the first latent space representation and the second latent space representation based on correlation among latent space representations; and
a final classifier engine to determine a characteristic of a user based on the weighted first and second latent space representations.

2. The system of claim 1, wherein the attention engine is to apply a first weight to the first latent space representation, the first weight larger than a second weight applied to the second latent space representation, based on the first latent space representation being more highly correlated to other latent space representations than the second latent space representation.

3. The system of claim 1, further comprising a pre-processing engine to convert the first signal to a first time series, and a feature extraction engine to determine a first feature vector based on the first time series.

4. The system of claim 1, further comprising a third classifier engine to concatenate a third feature vector from a third biometric sensor with a fourth feature vector from a fourth biometric sensor and to produce a third latent space representation based on the concatenation of the third feature vector and the fourth feature vector, wherein the attention engine is to weight the third latent space representation, and wherein the final classifier engine is to determine the characteristic based on the weighted third latent space representation.

5. The system of claim 1, further comprising a head-mounted display, wherein the system is to alter an audio or video output by the head-mounted display based on the determined characteristic of the user.

6. A method, comprising:
measuring a first biometric signal and a second biometric signal from a user of a head-mounted display;
generating a first latent space representation based on the first biometric signal;
generating a second latent space representation based on the second biometric signal;
weighting the first latent space representation and the second latent space representation based on correlations among latent space representations;
determining a characteristic of the user based on the weighted first and second latent space representations;
modifying audio or video content based on the determined characteristic; and
delivering the modified audio or video content to the user of the head-mounted display.

7. The method of claim 6, further comprising training a first classifier to determine the characteristic based on the first biometric signal and training a second classifier to determine the characteristic based on the second biometric signal, wherein generating the first latent space representation comprises generating the first latent space representation using the first classifier, and wherein generating the second latent space representation comprises generating the second latent space representation using the second classifier.

8. The method of claim 7, wherein the first and second classifiers include softmax functions to produce the first and second latent space representations, and wherein the method further comprises computing the correlation between the first latent space representation and the second latent space representation.

9. The method of claim 6, wherein determining the characteristic of the user includes determining a cognitive load of the user.

10. The method of claim 9, wherein modifying the audio or video content comprises modifying the audio or video content to cause an increase or decrease in the cognitive load of the user toward a predetermined cognitive load.

11. A non-transitory computer-readable medium comprising instructions that, when executed by a processor, cause the processor to:
generate a first latent space representation indicative of a characteristic of a user based on a first signal from a first biometric sensor;
generate a second latent space representation indicative of the characteristic of a user based on a second signal from a second biometric sensor;
generate a third latent space representation indicative of the characteristic of a user based on a third signal from a third biometric sensor calculate correlations between the first, second, and third soft latent space representations;
weight each of the first, second, and third latent space representations based on the correlations of that latent space representation with the other latent space representations; and
determine the characteristic of a user based on the weighted first, second, and third latent space representations.

12. The computer-readable medium of claim 11, wherein the instructions to calculate the correlations include instructions that cause the processor to stack the first, second, and third latent space representations to form a matrix, multiply the matrix by its transpose to produce a correlation matrix, and scale the correlation matrix to produce a scaled correlation matrix.

13. The computer-readable medium of claim 11, wherein the instructions to weight each of the first, second, and third latent space representations include instructions that cause the processor to multiply the scaled correlation matrix with the matrix.

14. The computer-readable medium of claim 11, wherein the first latent space representation is a soft determination calculated by a classifier based on the first signal.

15. The computer-readable medium of claim 11, further comprising instructions to further weight each of the weighted first, second, and third latent space representations based on values of that representation, wherein the instructions to determine the characteristic comprise instructions that cause the processor to determine the characteristic based on the further weighted first, second, and third latent space representations.
